(19) United States
(12) Patent Application Publication
(10) Pub. No.: US 2026/0102279 A1
(43) Pub. Date: Apr. 16, 2026

(54) Transparent Pad

(71) Applicant: C. R. Bard, Inc., Franklin Lakes, NJ (US)

(72) Inventors: Cecille A. Canary, Atlanta, GA (US); Nathaniel Barnes, Covington, GA (US); Christopher K. Brooks, Lawrenceville, GA (US); Younggeun Cho, Covington, GA (US); Sean E. Walker, Platteville, CO (US)

(21) Appl. No.: 19/420,487

(22) Filed: Dec. 15, 2025

Related U.S. Application Data
(63) Continuation of application No. 17/583,090, filed on Jan. 24, 2022, now Pat. No. 12,496,215.
(60) Provisional application No. 63/141,358, filed on Jan. 25, 2021.

(51) Int. Cl. A61F 7/02; A61F 7/00

(52) U.S. Cl. CPC A61F 7/02; A61F 7/0085; A61F 2007/0054; A61F 2007/0247

(57) ABSTRACT

Disclosed herein is a medical pad for exchanging thermal energy between a targeted temperature management (TTM) fluid and a patient. The pad includes a top side and a bottom side, as well as a fluid containing layer disposed between the top side and the bottom side, an insulation layer disposed on the top side, and a thermal conduction layer disposed on the bottom side. The pad can be configured to inhibit irritation of the skin of the patient when the pad is applied to the skin. The pad can also include translucent portions to facilitate visual observance of the skin beneath the pad.

### PRIORITY

This application is a continuation of U.S. patent application Ser. No. 17/583,090, filed Jan. 24, 2022, now U.S. Pat. No. 12,496,215, which claims the benefit of priority to U.S. Provisional Application No. 63/141,358, filed Jan. 25, 2021, each of which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems include a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001 and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain thermally intimate pad-to-patient contact is of importance to fully realizing medical efficacies with TTM systems.

In some instances, the application of a medical device such as a pad, for example, may cause some irritation of the patient's skin. In some instances, irritation may be caused by abrasion of the skin by the pad, particularly along a perimeter of the pad. In other instances, irritation of the skin may be caused by reduced breathability of the skin. In some instances, it may be advantageous for the clinician to visually observe the skin beneath the pad to assess the state of irritation so that corrective action may be taken to resolve the irritation. In a case of TTM therapy, it may be advantageous for the clinician to assess the state irrational without disrupting the TTM therapy. In summary, TTM systems and methods that minimize skin irritation and/or provide for easy assessment of skin irritation may reduce complications of performing the TTM therapy and reduce discomfort of the patient during TTM therapy. Disclosed herein are embodiments of devices and methods for resolving skin irritation while performing TTM therapy.

### SUMMARY OF THE INVENTION

Briefly summarized, disclosed herein is a medical pad for exchanging thermal energy between a targeted temperature management (TTM) fluid and a patient. The pad includes a top side and a bottom side. The pad further includes a fluid containing layer disposed between the top side and the bottom side, wherein the fluid containing layer is configured for containing the TTM fluid. The fluid containing layer includes a fluid inlet and a fluid outlet and the TTM fluid is circulatable within the fluid containing layer from the fluid inlet to the fluid outlet. The pad further includes an insulation layer disposed the top side, and a thermal conduction layer disposed on the bottom side. In use, the pad is disposed in contact with a skin of a patient, and a perimeter of the pad is configured to inhibit irritation of the skin of the patient along the perimeter.

The pad may further include a chamfered edge extending along the perimeter of the pad, and in some embodiments, chamfered edge is a top edge of the pad. The pad may also include a rounded edge extending along the perimeter of the pad. The rounded edge may include a tube extending along the perimeter, a wall of the tube extends from the top side to the bottom side of the pad.

In some embodiments, the thermal conduction layer includes a skin contact surface configured to provide for breathability of the skin and the skin contact surface may be textured.

In some embodiments, the pad is configured to provide for visibility of the skin through the pad. At least a portion of the pad may be translucent. Each of the fluid containing layer and the thermal conduction layer may include a translucent portion, and the translucent portions may be coincident with each other. Each of the translucent portions may also be transparent.

The insulation layer may include at least one of an opening or translucent portion which may be disposed coincident with the translucent portions of the fluid containing layer and the thermal conduction layer.

The translucent portion of the insulation layer may include one or more air pockets, and in some embodiments, the translucent portion of the insulation layer is transparent.

In some embodiments, the pad includes a filter coupled to the fluid containing layer so that TTM fluid circulating through the fluid containing layer passes through the filter and the filter may include a porous wall disposed parallel to a continuous flow path through the filter.

Also disclosed herein is a method of providing a targeted temperature management (TTM) therapy to a patient. The method includes providing a TTM system including a TTM module configured to provide a TTM fluid, a thermal pad configured to receive the TTM fluid from the TTM module to facilitate thermal energy transfer between the TTM fluid and a patient, and a fluid delivery line (FDL) extending between the TTM module and the thermal pad, the FDL configured to provide TTM fluid flow between the TTM module and the thermal pad.

The thermal pad includes a top side, bottom side, and a fluid containing layer disposed between the top side and the bottom side. The fluid containing layer is configured for containing the TTM fluid and includes a fluid inlet and a fluid outlet, where the TTM fluid is circulatable within the fluid containing layer from the fluid inlet to the fluid outlet. The thermal pad further includes an insulation layer disposed the top side and a thermal conduction layer disposed on the bottom side.

The method further includes applying the thermal pad to the patient, delivering the TTM fluid from the TTM module to the thermal pad, and visually observing the skin through a translucent portion of the thermal pad. The method may further include passing TTM fluid through a filter coupled to the fluid containing layer. In some embodiments, the thermal pad further includes a chamfered edge extending along a perimeter of the thermal pad. In other embodiments, the thermal pad further includes a rounded edge extending along a perimeter of the thermal pad. The thermal conduction layer may include a textured bottom surface to facilitate breathability of the skin beneath the thermal pad. In some embodiments, at least a portion of the thermal pad is translucent and the method further includes visually observing the skin through the translucent portion.

Also disclosed herein is a medical pad for exchanging thermal energy between a targeted temperature management (TTM) fluid and a patient. The pad includes a top side and a bottom side. The pad further includes a fluid containing layer disposed between the top side and the bottom side, wherein the fluid containing layer is configured for containing the TTM fluid. The fluid containing layer includes a fluid inlet and a fluid outlet and the TTM fluid is circulatable within the fluid containing layer from the fluid inlet to the fluid outlet. The pad further includes an insulation layer disposed the top side, and a thermal conduction layer disposed on the bottom side. In use, the pad is disposed in contact with a skin of a patient, and the pad is configured to provide for visibility of the skin through the pad.

At least a portion of the pad may be translucent. Each of the fluid containing layer and the thermal conduction layer may include a translucent portion, and the translucent portions may be coincident with each other. Each of the translucent portions may also be transparent.

The insulation layer may include at least one of an opening or translucent portion which may be disposed coincident with the translucent portions of the fluid containing layer and the thermal conduction layer.

The translucent portion of the insulation layer may include one or more air pockets, and in some embodiments, the translucent portion of the insulation layer is transparent.

The pad may further include a chamfered edge extending along the perimeter of the pad, and in some embodiments, chamfered edge is a top edge of the pad. The pad may also include a rounded edge extending along the perimeter of the pad. The rounded edge may include a tube extending along the perimeter, a wall of the tube extends from the top side to the bottom side of the pad.

In some embodiments, the thermal conduction layer includes a skin contact surface configured to provide for breathability of the skin and the skin contact surface may be textured.

In some embodiments, the pad includes a filter coupled to the fluid containing layer so that TTM fluid circulating through the fluid containing layer passes through the filter and the filter may include a porous wall disposed parallel to a continuous flow path through the filter.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a targeted temperature management (TTM) system for cooling or warming a patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4A is a top view of the thermal contact pad of FIG. 1, in accordance with some embodiments.

FIG. 4B is a cross-sectional side view of the thermal contact pad of FIG. 4A cut along sectioning lines 4B-4B, in accordance with some embodiments.

FIG. 4C is a detail cross-sectional side view of a portion of the thermal contact pad of FIG. 4A cut along sectioning lines 4C-4C illustrating an optional chamfered edge, in accordance with some embodiments.

FIG. 4D is a detail cross-sectional side view of a portion of the thermal contact pad of FIG. 4A cut along sectioning lines 4C-4C illustrating an optional rounded edge, in accordance with some embodiments.

FIG. 4E is a bottom perspective view of thermal contact pad of FIG. 4A illustrating a textured bottom surface, in accordance with some embodiments.

FIG. 5A is a top perspective view of an embodiment of the insulation layer of FIG. 4B, in accordance with some embodiments.

FIG. 5B is a top perspective view of another embodiment of the insulation layer of FIG. 4B, in accordance with some embodiments.

FIG. 6A is an exploded perspective view of a TTM fluid filter, in accordance with some embodiments.

FIG. 6B is a cross-sectional side view of the filter of FIG. 6A, in accordance with some embodiments.

FIG. 6C is a side cross-sectional view of the thermal contact pad of FIG. 1 incorporating the filter of FIG. 6A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

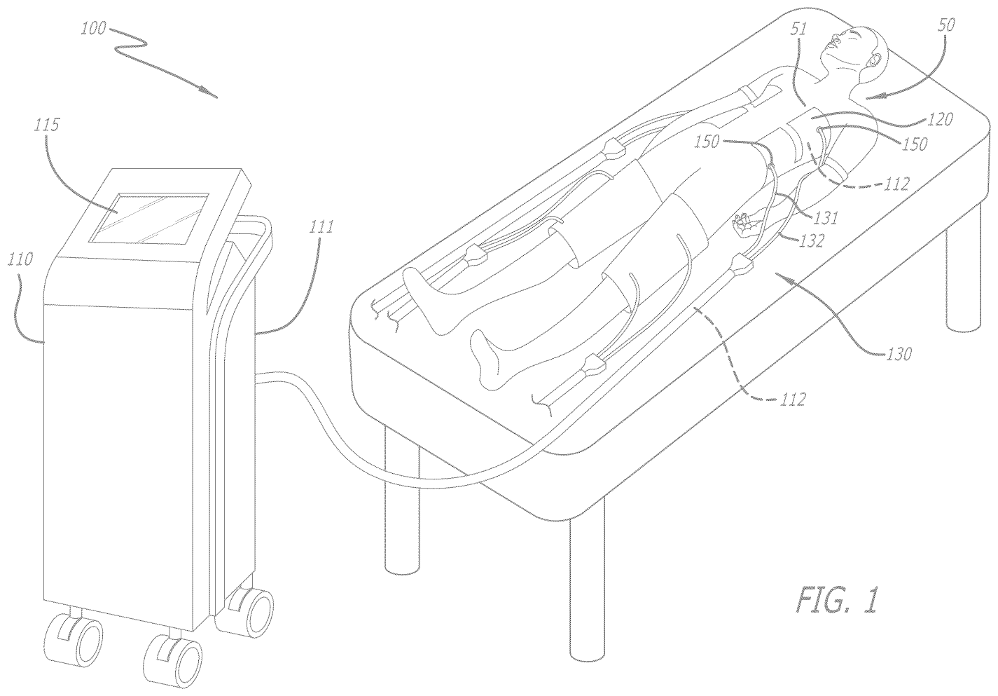

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 includes a TTM module 110 including a graphical user interface (GUI) 115 enclosed within a module housing 111. The TTM system 100 includes a fluid deliver line (FDL) 130 extending from the TTM module 110 to a thermal contact pad 120 to provide for flow of TTM fluid 112 between the TTM module 110 and the pad 120. The FDL includes two conduits to facilitate delivery flow of TTM fluid 112 from the TTM module 110 to the pad 120 and return flow TTM fluid 112 from the pad 120 to the TTM module 110. In some embodiments, the two conduits may be attached to each other along a portion of a length of the FDL.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 along a TTM fluid flow path including within the pad 120. The pad 120 is applied to the skin 51 of the patient to facilitate thermal energy exchange between the pad 120 and the patient 50. During the TTM therapy, the TTM module 110 may continually control the temperature of the TTM fluid 112 toward a target TTM temperature.

The FDL 130 includes at least a fluid delivery conduit 131 and a fluid return conduit 132. In use, the TTM fluid 112 may flow from the TTM module 110 through the fluid delivery conduit 131 to the pad 120. The TTM fluid 112 may then flow from thermal pad 120 through the fluid return conduit 132 to the TTM module 110. In some embodiments, the fluid delivery conduit 131 and the fluid return conduit 132 may be attached together along a portion of a length of the FDL 130. The fluid delivery conduit 131 and the fluid return conduit 132 may be separated from each other at each end of the FDL 130.

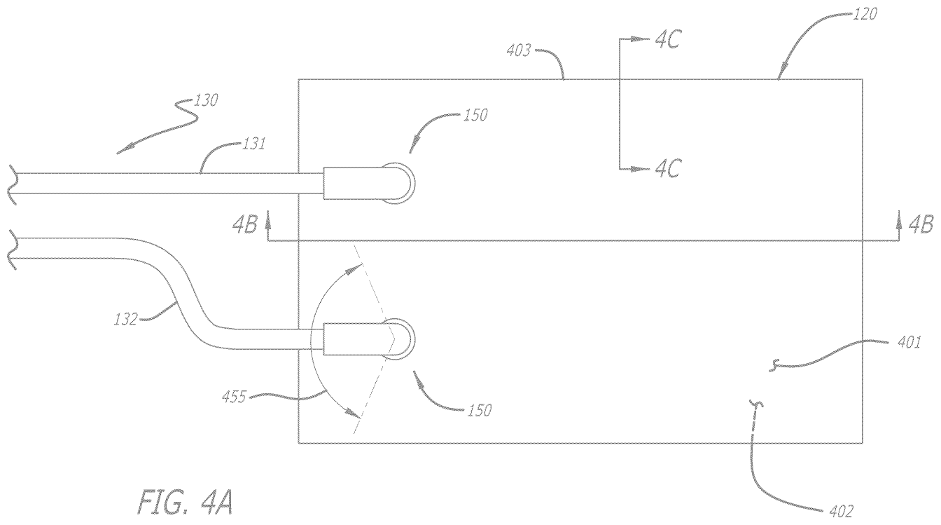

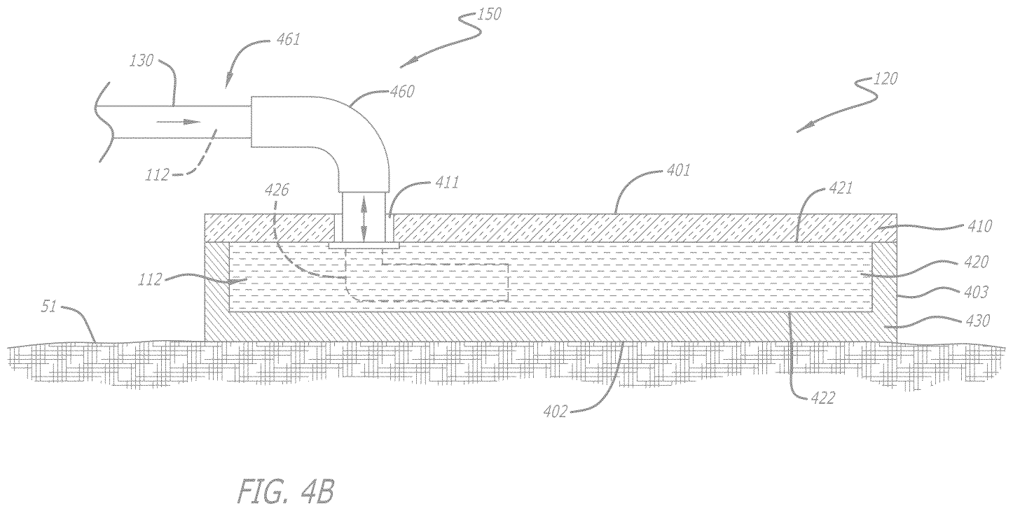

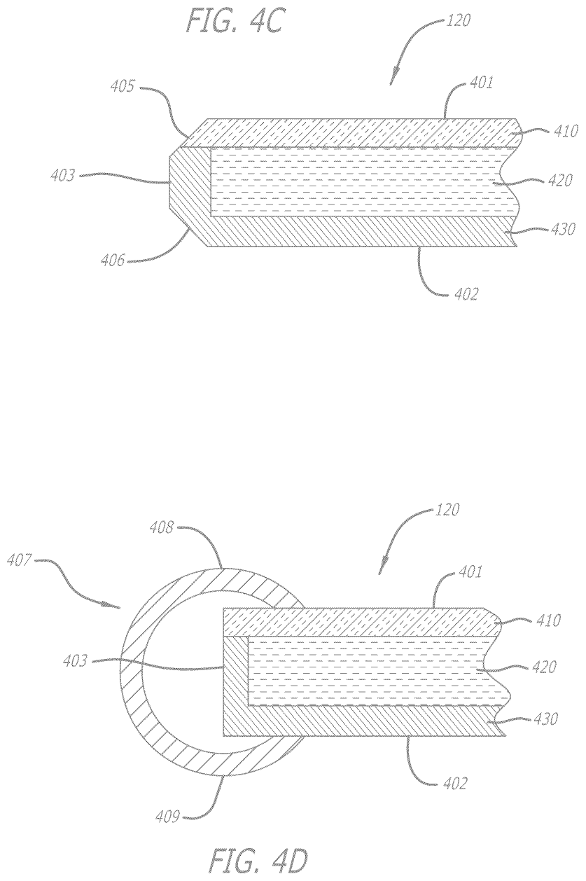

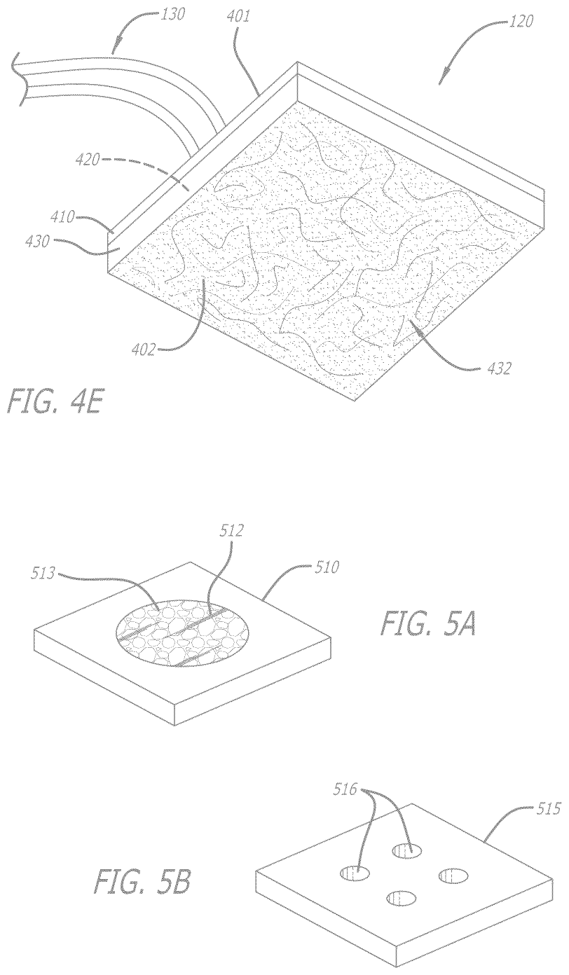

The TTM system 100 may include a connector system 150 to couple the FDL 130 to the pad 120. In some embodiments, the connector system 150 may couple a single fluid conduit of the FDL to the pad 120. Hence, the connection between the FDL 130 and the pad 120 may include more than one connector system 150 to couple more than one fluid conduit to the pad 120. The connector system 150 is further described below in FIGS. 4A and 4B.

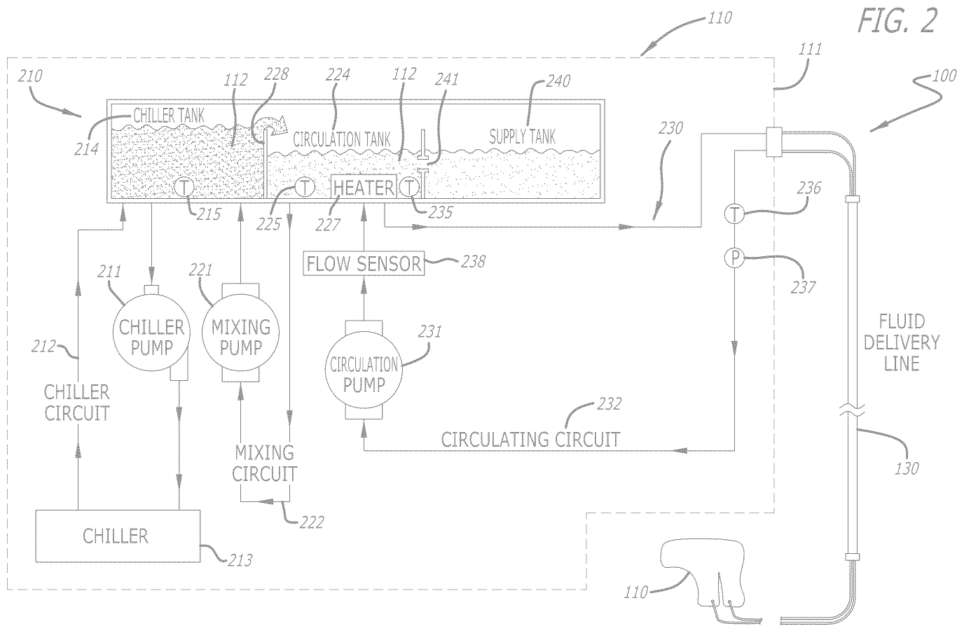

FIG. 2 illustrates a hydraulic schematic of the TTM system 100. The FDL 130 and the pad 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

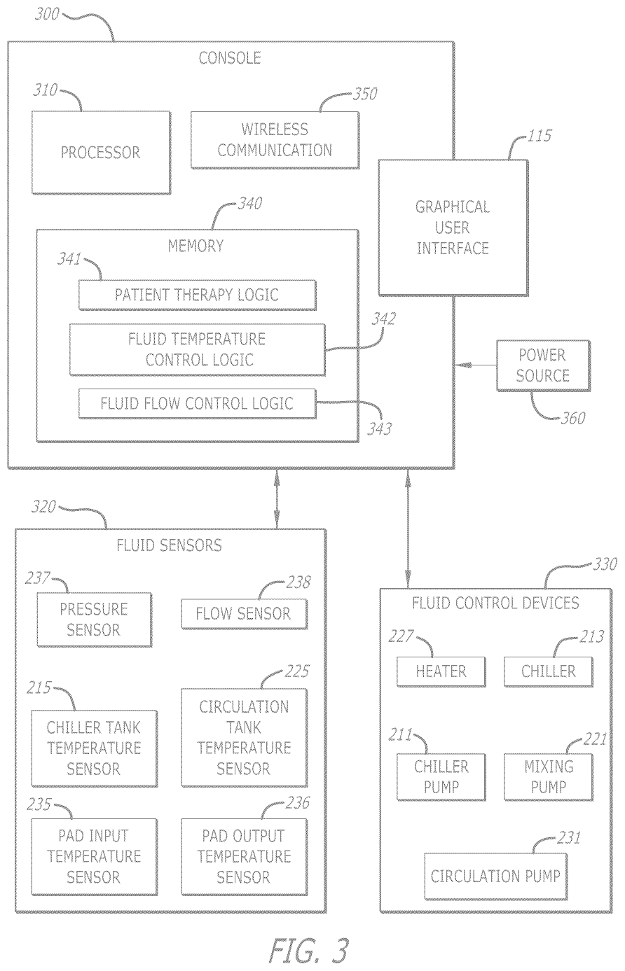

The temperature control subsystem 210 may include a chiller pump 211 to pump (recirculate) TTM fluid 112 through a chiller circuit 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may 1) receive temperature data from the temperature sensor 215 within the chiller tank and the temperature sensor 225 within the circulation tank 224 and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 includes a circulation pump 213 to pull TTM fluid 112 from the circulation tank 224 and through a circulating circuit 232 that includes the fluid delivery line 130 and the pad 120 located upstream of the circulation pump 213. The circulating circuit 232 also includes a pressure sensor 237 to represent a pressure of the TTM fluid 112 within the pad 120. The circulating circuit 232 includes a temperature sensor 235 within the circulation tank 224 to represent the temperature of the TTM fluid 112 entering the pad 120 and a temperature sensor 236 to represent the temperature of the TTM fluid exiting the pad 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation than) the pad 120 so that a pressure within the pad 120 is less than atmospheric pressure (i.e., negative) when TTM fluid flow through the circulating circuit 232 is stopped. The pad 120 is also placed upstream of the circulation pump 231 to further establish a negative pressure within the pad 120 when the circulation pump 213 is operating. The fluid flow control logic (see FIG. 3) may control the operation of the circulation pump 213 to establish and maintain a desired negative pressure within the pad 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module 110 includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 and/or a thermal energy exchange rate which may include a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad 120 in accordance with the predefined target temperature. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224 by mixing TTM fluid 112 from the chiller tank 214 with TTM fluid 112 within circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad 120, the fluid flow control logic 343 may, in some embodiments, control the operation of the circulation pump 231 in accordance with a defined thermal energy exchange rate for the TTM therapy.

The console 300 may include or be couple do wireless communication module 350 to facilitate wireless communication with external devices. A power source 360 provides electrical power to the console 300.

FIG. 4A shows a top view of the pad 120 in accordance with some embodiments. The pad 120 includes a top surface 401, bottom surface 402, and an outside edge 403 extending along a circumference of the pad 120. In the illustrated embodiment, the pad 120 includes two connector systems 150 coupled to the FDL 130. As illustrated, the connector systems 150 may provide for a rotatable connection between the FDL 130 and the pad 120. The rotatable connection may provide for the FDL 130, or more specifically each of the fluid delivery conduit 131 and the fluid return conduit 132, to rotate through an angle 455 ranging up to about 90 degrees, 180 degrees, or 360 degrees.

FIG. 4B shows a cross-sectional side view of the pad 120 in contact with the skin 51, in accordance with some embodiments. As shown, the connector system 150 may include an elbow 460 to change the direction of FDL 130 extending away from the connector system 150. As shown, the direction of FDL 130 is shifted from a direction perpendicular to the pad 120 to a direction that is substantially parallel to the pad 120. The elbow 450 also establishes an orientation of a distal portion 461 of the FDL 130 to be substantially parallel to the pad 120 and/or the fluid containing layer 420. The fluid containing layer 420 may include one or more internal fluid conduits 426 fluidly coupled to the FDL 130 and the TTM fluid 112 may flow through the internal fluid conduits 426.

The pad 120 includes multiple layers to provide for multiple functions of the pad 120. A fluid containing layer 420 is shown fluidly coupled to the fluid delivery conduit 131 of the FDL 130 to facilitate circulation of the TTM fluid 112 within the fluid containing layer 420. The fluid containing layer 420, having TTM fluid 112 circulating therein, defines a heat sink or a heat source for the patient 50 in accordance with a temperature of the TTM fluid 112. The fluid containing layer 420, or a portion thereof, may be formed of translucent materials so that the fluid containing layer 420, or the portion thereof, is translucent from the top side 421 to the bottom side 422. In other embodiments, the fluid containing layer 420, or a portion thereof, may be formed of transparent materials so that the fluid containing layer 420, or the portion thereof, is transparent from the top side 421 to the bottom side 422.

The pad 120 includes a thermal conduction layer 430 disposed between the fluid containing layer 420 and the patient's skin 51. The thermal conduction layer 430 is configured to facilitate thermal energy exchange between the fluid containing layer 420 and the patient 50. The thermal conduction layer 430 may be attached to the fluid containing layer 420 along a bottom surface 421 of the fluid containing layer 420. The thermal conduction layer 430 may be conformable to provide for intimate thermal contact with the patient 50. In other words, thermal conduction layer 430 may conform to a contour of the patient 50 to facilitate thermal energy exchange between the thermal conduction layer 430 and the patient 50.

The thermal conduction layer 430, or a portion thereof, may be formed of translucent materials so that the thermal conduction layer 430, or the portion thereof, is translucent from the bottom side 422 of the fluid containing layer 420 to the bottom side 402 of the pad 120. In other embodiments, the thermal conduction layer 430, or a portion thereof, may be formed of transparent materials so that the thermal conduction layer 430, or the portion thereof, is transparent from the bottom side 422 of the fluid containing layer 420 to the bottom side 402 of the pad 120.

The pad 120 includes an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may include one or more openings 411 extending through the insulation layer 410 to provide for coupling of the FDL 130 with the fluid containing layer 420.

The insulation layer 410, or a portion thereof, may be formed of translucent materials so that the insulation layer 410, or the portion thereof, is translucent from the top side 401 of the pad 120 to the top side 421 of the fluid containing layer 420. In other embodiments, the insulation layer 410, or a portion thereof, may be formed of transparent materials so that the insulation layer 410, or the portion thereof, is transparent from the top side 401 of the pad 120 to the top side 421 of the fluid containing layer 420.

FIG. 4C is a cross-sectional side view of an embodiment of the pad 120 cut along sectioning lines 4C-4C. In the illustrated embodiment, the pad 120 includes a top chamfer 405 and a bottom chamfer 406 extending along a perimeter of the pad 120. The top chamfer 405 and the bottom chamfer 406 may add flexibility to the pad 120 along the perimeter to reduce pressure contact points and/or abrasion contact points of the pad 120 on the skin 51. As such, the top chamfer 406 and/or the bottom chamfer 405 may inhibit skin irritation of the patient 50 along the perimeter of the pad 120. The top chamfer 406 and/or the bottom chamfer 405 may also allow the clinician to visually observe the skin 51 along the perimeter beneath the pad 120. In some embodiments, the top chamfer 405 and a bottom chamfer 406 may extend around the entire circumference of the pad 120. In other embodiments, the top chamfer 405 and a bottom chamfer 406 may extend along one or more perimeter sections of the circumference. In some embodiments, either one of the top chamfer 405 or the bottom chamfer 406 may be omitted.

FIG. 4D is a cross-sectional side view of another embodiment of the pad 120 cut along sectioning lines 4C-4C. In this illustrated embodiment, the pad 120 includes a rounded perimeter edge 407 of the pad 120. The rounded perimeter edge 407 may include a tube 408 having a wall 409 that is cut lengthwise along a length of the tube 408. The tube 408 may be attached to the pad 120 such that the wall 409 extends around the outside edge 403 of the pad 120 from the top side 401 to the bottom side 402 of the pad 120. The rounded edge 407 may reduce pressure contact points and/or abrasion contact points of the pad 120 on the skin 51. As such, the rounded perimeter edge 407 may inhibit skin irritation of the patient 50 along the rounded perimeter edge 407. In some embodiments, rounded perimeter edge 407 may extend around the entire circumference of the pad 120. In other embodiments, the rounded perimeter edge 407 may extend along one or more perimeter sections of the circumference.

FIG. 4E is a perspective bottom view of the pad 120 showing a textured bottom surface 432 of the thermal conduction layer 430. The textured bottom surface 432 may be configured to provide for breathability of the skin 51 when the pad 120 is applied to the skin 51. The textured bottom surface 432 may facilitate breathability of the skin 51 while maintaining thermal energy exchange between fluid containing layer 420 and the patient 50. The bottom surface 432 may include multiple protrusions and/or depressions that provide for airflow between the skin 51 and the thermal conduction layer 630. The protrusions may include any structure extending away from the surface, such as ribs or bumps, for example. The depressions may include any structure that is depressed from surface such as troughs or dimples, for example. In some embodiments, the textured bottom surface 432 may include a layer component (e.g., a fabric mesh) to provide for breathability of the skin 51.

FIG. 5A is a top perspective view of an insulation layer 510 that may be included with the pad 120. The insulation layer 510 can, in certain respects, resemble components of the insulation layer 410. It will be appreciated that all the illustrated embodiments may have analogous features. Relevant disclosure set forth above regarding similarly identified features thus may not be repeated hereafter. However, such features may clearly be the same, or substantially the same, as features depicted in other embodiments and/or described with respect to such embodiments.

The insulation layer 510 includes a translucent portion 512. The translucent portion 512 may be formed of translucent materials and may include multiple air pockets 513 (e.g., enclosed in translucent plastic) that establish thermal insulative properties of the insulation layer 510 while maintaining translucence. In some embodiments, the translucent portion 512 may include the entire insulation layer 510. In other embodiments, the insulation layer 510 may include multiple translucent portions 512. The translucent portion 512 may, in combination with the translucence of the fluid containing layer 420 and the thermal conduction layer 430, provide for visibility of the skin 51 through the pad 120. In other embodiments, the translucent portion 512 may be transparent (e.g., the multiple air pockets 513 enclosed within transparent plastic) so that in combination with the transparency of the fluid containing layer 420 and the thermal conduction layer 430, the pad 120 may provide for transparent visibility of the skin 51 through the pad 120.

FIG. 5B is a top perspective view of an insulation layer 515 that may be included with the pad 120. The insulation layer 515 includes one or more openings 516 extending through the insulation layer 515. The openings 516 may be configured to provide for visibility through the insulation layer 515. The one or more openings 516 may, in combination with the translucence of the fluid containing layer 420 and the thermal conduction layer 430, provide for visibility of the skin 51 through the pad 120.

By way of summary, in some embodiments, at least a portion of each layer of the pad 120 may be configured for visibility therethrough. In such embodiments, the pad 120 may be formed so that the individual visibility portions of the layers are aligned coincidently with each other. As such, at least a portion of the pad 120 may be configured for translucent and/or transparent visibility therethrough.

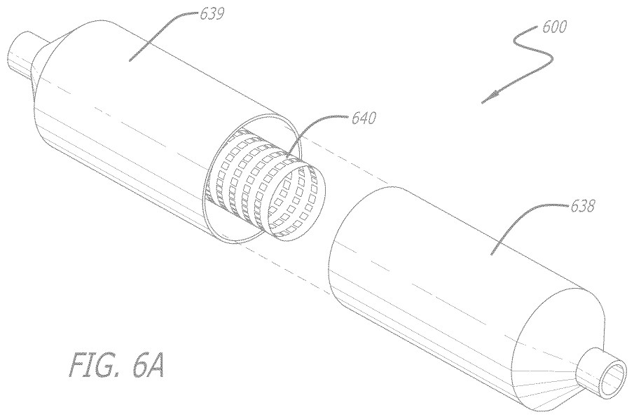

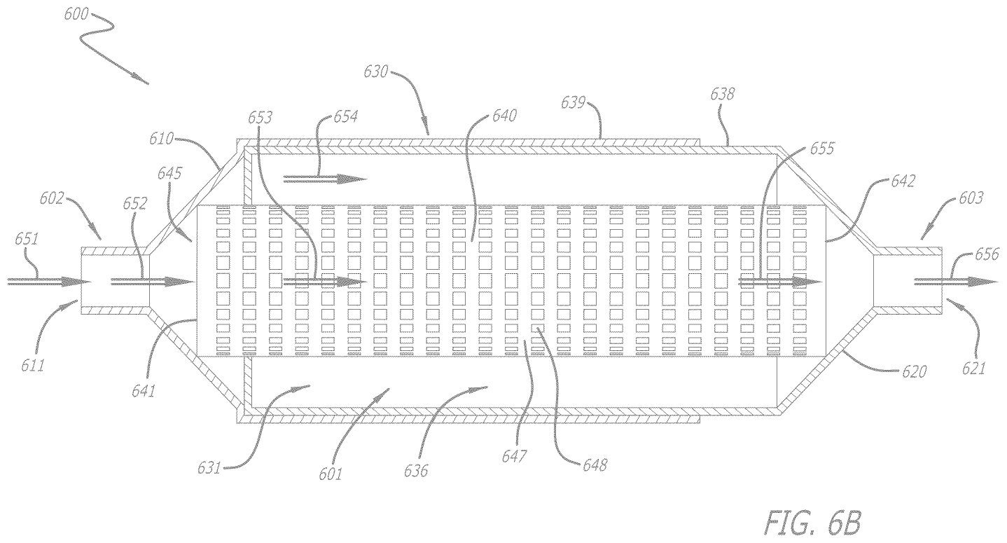

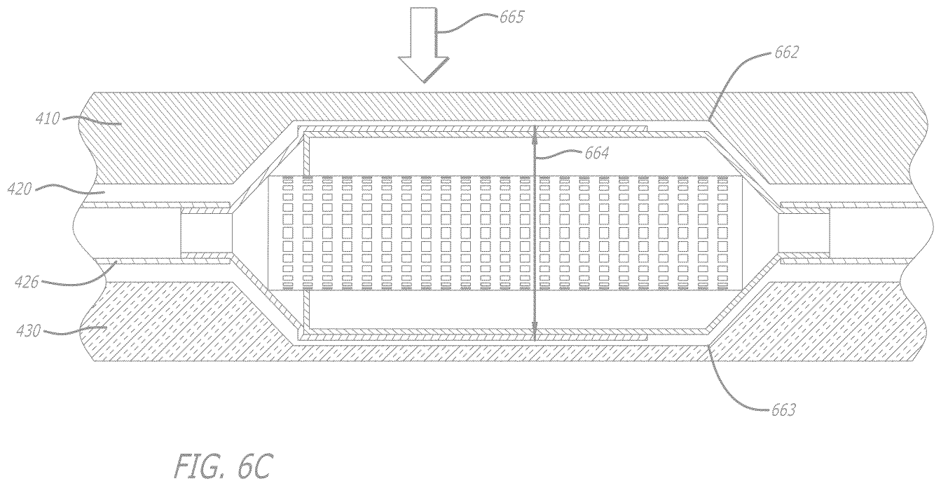

FIGS. 6A and 6B show a filter 600 that may be included with the TTM system 100. The filter 600 may be disposed in line with a TTM fluid flow path of the TTM system 100 so that the circulating TTM fluid 112 flows through the filter 600. The filter 600 may be configured to remove (i.e., filter out) material/particles having a size of 0.2 microns or larger from the TTM fluid 112 without causing a flow restriction of the TTM fluid 112.

The filter 600 includes a longitudinal shape having a flow path 601 extending from a first end 602 to a second end 603. The filter 600 includes a diffuser 610 adjacent the first end 602, a nozzle adjacent 620 the second end 603, and a body 630 extending between the diffuser 610 and the nozzle 620. Along the diffuser 610, a cross-sectional flow area of the filter 600 expands from an inlet flow area 611 to a body flow area 631 and along the nozzle 620, the cross-sectional flow area of the filter 600 contracts from the body flow area 631 to an outlet flow area 621. In some embodiments, the inlet flow area 611 and the outlet flow area 621 may be substantially equal.

In some embodiments, the body flow area 631 may be constant along the body 630. In other embodiments, the body flow area 631 may vary along a length of the body 630 such that the body flow area 631 is greater or less along middle portion of the body 630 than at the ends of the body 630. In some embodiments, the body flow area 631 may be circular.

The filter 600 includes an inner tube 640 disposed within the body 630 extending along the length of body 630. The inner tube 640 may be coupled to the diffuser 610 at a first inner tube end 641 so that TTM fluid 112 entering the filter 600 at the first end 602 also enters the inner tube 640 at the first inner tube end 641. The inner tube 640 may be coupled to the nozzle 620 at a second inner tube end 642 so that TTM fluid 112 exiting the filter 600 at the second end 603 also exits the inner tube 640 at the second inner tube end 642.

The inner tube 640 includes an inner tube flow area 645 extending the length of the inner tube 640. The inner tube flow area 645 may be greater than the inlet flow area 611 and/or the outlet flow area 621. The inner tube flow area 645 may be constant along the length of the inner tube 640. In some embodiments, the inner tube flow area 645 may vary along the length of the inner tube 640. In some embodiments, the inner tube 640 may include a circular cross section. The inner tube 640 and the body 630 may be configured so that the body flow area 631 includes a combination of the inner tube flow area 645 and an annular flow area 636.

The inner tube 640 includes a porous a circumferential wall 647. The porous wall 647 may be configured so that TTM fluid 112 may flow through the porous wall 647, i.e., through the pores 648 of the porous wall 647. Consequently, TTM fluid 112 may flow through the porous wall 647 from the inner tube flow area 645 to the annular flow area 636 and from the annular flow area 636 into the inner tube flow area 645.

In use, the longitudinal velocity of the TTM fluid 112 may change along the length of the filter 600. As the volumetric TTM fluid 112 flow through the filter is constant, the longitudinal velocity of the TTM fluid 112 may be at least partially defined by the flow areas of the filter 600 as described below. The TTM fluid 112 may enter the filter 600 at a first longitudinal velocity 651 and decrease along the diffuser so that the TTM fluid 112 enters the inner tube at a second velocity 652 less than the first longitudinal velocity 651. At this point, a portion of the TTM fluid 112 may flow through the porous wall 647 from the inner tube flow area 645 into the annular flow area 636 to divide the fluid flow into a third velocity 653 within the inner tube flow area 645 and a fourth velocity 654 within the annular flow area 636. The fourth velocity 654 may be less than the third velocity 653. A portion of the TTM fluid 112 may then flow back into the inner tube flow area 645 from the annular flow area 636 to define a fifth velocity 655 along the inner tube flow area 645 which may be about equal to the second velocity 652. The TTM fluid 112 may then proceed along the nozzle 620 to define a sixth velocity 656 exiting the filter 600. In some embodiments, the first velocity 651 and the sixth velocity 656 may be about equal.

The filter 600 may be configured to remove harmful bacteria and viruses from the TTM fluid 112 using sedimentation principles. In use, the filter 600 may be oriented horizontally so that the direction of fluid flow through the filter 600 is perpendicular to a gravitational force 665. In some instances, bacteria, viruses, and other particles within the TTM fluid 112 may have a greater density than the TTM fluid 112 and as such may be urged by the gravitational force 665 (i.e., sink) in a direction perpendicular to the fluid flow direction. In some instances, particles within the inner tube flow area 645 may sink toward and through the porous wall 647 into the annular flow area 636. Particles within the annular flow area 636 may then sink toward an inside surface 631 of the body 630 and become trapped adjacent the inside surface 631. The geometry of the filter 600 may be configured to allow 0.2-micron bacteria/virus particles to fall out of the flow of TTM fluid 112 and become trapped along the inside surface 631.

In some embodiments, the filter 600 may be configured so that flow of TTM fluid 112 from the inner tube flow area 645 into the annual flow area 636 my drag particles through the porous wall 647. In some embodiments, the inlet flow area 611, the inner tube flow area 645, and the annual flow area 636 may be sized so that the third velocity 1053 is less than about 50 percent, 25 percent, or 10 percent of the first velocity 651 or less. In some embodiments, the body 630 and the inner tube 640 may be configured so that the fourth velocity 654 is less than the third velocity 653. In some embodiments, the fourth velocity 654 may less than about 50 percent, 25 percent, or 10 percent of the third velocity 653 or less.

In some embodiments, the filter 600 may be configured so that the flow within the inner tube flow area 645 is laminar flow, i.e., so that the velocity of the fluid flow adjacent to or in close proximity to an inside surface 641 of the porous wall 647 is less than the velocity at a location spaced away from the inside surface 641. In such an embodiment, the particles may more readily sink toward and through the porous wall 647.

In some embodiments, the filter 600 may be configured so that the fluid flow within the annual flow area 636 is laminar flow, i.e., so that the velocity of the fluid flow adjacent to or in close proximity to inside surface 631 of the body 630 is less than the velocity at a location spaced away from the inside surface 631. In such an embodiment, the particles may more readily sink toward and be trapped along the inside surface 631.

The filter 600 may include three components including the inner tube 640 an inner body shell 638, and an outer body shell 639. Each of the three components may be formed via the plastic injection molding process. Assembly of the filter 600 may include capturing the inner tube 640 within the inner body shell 638 and the outer body shell 639 and sliding the inner body shell 638 into the outer body shell 639 wherein the fit between the inner body shell 638 and the outer body shell 639 is an interference fit.

In some embodiments, the filter 600 may be disposed within the pad 120. FIG. 6C shows a detail cross-sectional view of the pad 120 including the filter 600 disposed within the fluid containing layer 420. The filter 600 is coupled in line with the internal fluid conduit 426 within the fluid containing layer 420 so that TTM fluid 12 circulating within the pad 120 passes through the filter 600. The filter 600 may be sized so that the inlet flow area 611 and the outlet flow area 621 are similar to a cross-sectional flow area of the internal flow path 660 within the fluid containing layer 420.

In some embodiments, a thickness of the fluid containing layer 420 may increase adjacent the filter 600 to accommodate a body diameter 664 of the filter 600. To further accommodate the body diameter 664, the insulation layer 410 and/or the thermal conduction layer 430 may include internal depressions 662, 663, respectively.

In some embodiments, one or more filters 600 may be disposed in line with the flow of TTM fluid 112 at other locations of the TTM system 100. In some embodiments, one or more filters 600 may be disposed within the TTM module 110. In some embodiments, one or more filters 600 may be disposed in line with the FDL 130. In some embodiments, the filter 600 may be disposed in line with a fluid conduit of the pad external to the fluid containing layer 420 such as a conduit extending between the pad connector 652 and the pad 120.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A medical pad for exchanging thermal energy between a targeted temperature management (TTM) fluid and a patient, the medical pad comprising:
a perimeter configured to inhibit irritation of the patient;
a fluid containing layer disposed between a top side and a bottom side of the medical pad, wherein:
the fluid containing layer is configured for containing the TTM fluid,
the fluid containing layer comprises a fluid inlet and a fluid outlet, and
the TTM fluid is circulatable in the fluid containing layer from the fluid inlet to the fluid outlet; and
a thermal conduction layer disposed on the bottom side of the medical pad, wherein the fluid containing layer and the thermal conduction layer include one or more coincident translucent portions to enable visual observation of the patient.

2. The medical pad according to claim 1, further comprising an insulation layer disposed on the top side of the medical pad, the insulation layer including one or more translucent portions coincident with the one or more coincident translucent portions of the fluid containing layer and the thermal conduction layer.

3. The medical pad according to claim 1, further comprising at least one chamfered edge extending along the perimeter.

4. The medical pad according to claim 3, wherein the at least one chamfered edge is a top edge of the pad.

5. The medical pad according to claim 1, further comprising a rounded edge extending along the perimeter.

6. The medical pad according to claim 5, wherein the rounded edge comprises a tube extending along the perimeter, and wherein a wall of the tube extends from the top side to the bottom side of the medical pad.

7. The medical pad according to claim 1, wherein the thermal conduction layer comprises a skin contact surface configured to provide for breathability.

8. The medical pad according to claim 7, wherein the skin contact surface is textured.

9. The medical pad according to claim 1, wherein the one or more translucent portions of the insulation layer comprises one or more air pockets.

10. The medical pad according to claim 1, wherein the pad comprises a filter coupled to the fluid containing layer to enable TTM fluid circulating through the fluid containing layer to pass through the filter.

11. The medical pad according to claim 10, wherein the filter comprises a porous wall disposed parallel to a continuous flow path through the filter.
